(19) United States
(12) Patent Application Publication
(10) Pub. No.: US 2013/0281902 A1
(43) Pub. Date: Oct. 24, 2013

(54) Therapeutic finger brace device

(71) Applicant: Alex McCrae, Miramar, FL (US)

(72) Inventor: Alex McCrae, Miramar, FL (US)

(21) Appl. No.: 13/815,421

(22) Filed: Feb. 28, 2013

Related U.S. Application Data
(60) Provisional application No. 61/604,578, filed on Feb. 29, 2012.

(51) Int. Cl. A61F 5/01

(52) U.S. Cl. CPC A61F 5/0118
USPC 602/22

(57) ABSTRACT

A therapeutic finger brace having a main body defining a center aperture, a first arcuate end, a second arcuate end, an upper contoured side and a lower contoured side.

### CROSS REFERENCE TO RELATED APPLICATIONS

This application claims the benefit of provisional patent application Ser. No. 61/604,578 filed Feb. 29, 2012.

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

This invention relates generally to a therapeutic finger brace, and more particularly, to a therapeutic finger brace that trains fingers into their natural position as opposed to a collapsing position that impedes normal use of the hand.

2. Description of the Background Art

A variety of hand and finger braces are known in the art. However, there are no therapeutic finger or hand braces known that rehabilitate and train one's fingers to relax in a normal position after a serious injury or neurological disorder that causes them to overlap. Therefore, there exists a need for a finger or hand brace that retrains the fingers to assume their normal position. It is, therefore, to the effective resolution of the aforementioned problems and shortcomings of the prior art that the present invention is directed. The instant invention addresses this unfulfilled need in the prior art by providing a finger and hand brace as contemplated by the instant invention disclosed herein.

### BRIEF SUMMARY OF THE INVENTION

In light of the foregoing, it is an object of the present invention to provide a therapeutic finger brace device that rehabilitates a hand.

It is also an object of the instant invention to provide a therapeutic finger brace device that rehabilitates a hand by retaining the hand in a normal position.

It is another object of the instant invention to provide a therapeutic finger brace device that separates the fingers to effectuate rehabilitation.

It is an additional object of the instant invention to provide a therapeutic finger brace device that is simple in construction.

It is a further object of the instant invention to provide a therapeutic finger brace device that is cost effective to make.

In light of these and other objects, the instant invention comprises a therapeutic finger brace having a main body with a center aperture, a first arcuate end, a second arcuate end, an upper contoured side and a lower contoured side. The center aperture is designed for receiving a finger, such as the middle finger, while the first and second arcuate ends are adapted for receiving adjacent fingers. The upper contoured side and lower contoured side have an ergonomic design for comfort and facilitating use of the hand while wearing the therapeutic finger brace.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1 is an elevational view of the finger brace in accordance with the preferred embodiment of the instant invention.

FIG. 2 is a perspective view of the finger brace in accordance with the preferred embodiment of the instant invention.

FIG. 3 is a pictorial view of the finger brace as used in accordance with the preferred embodiment of the instant invention.

### DETAILED DESCRIPTION OF THE INVENTION

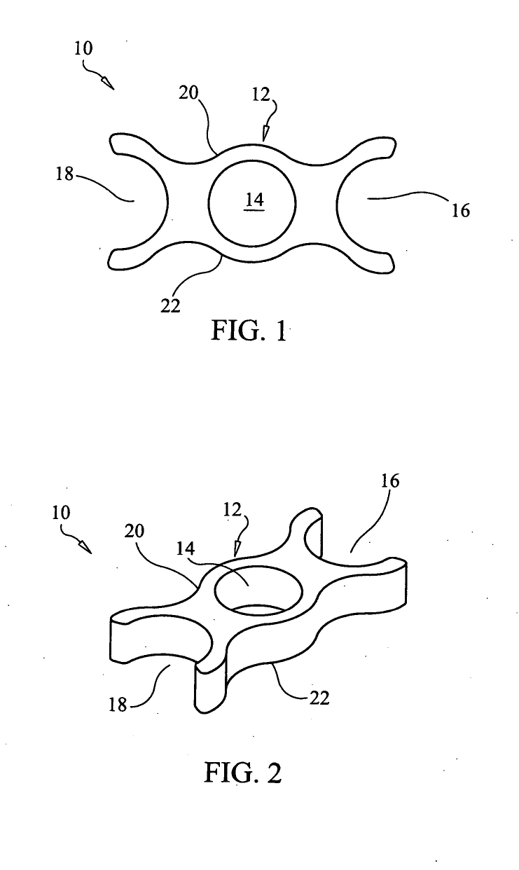

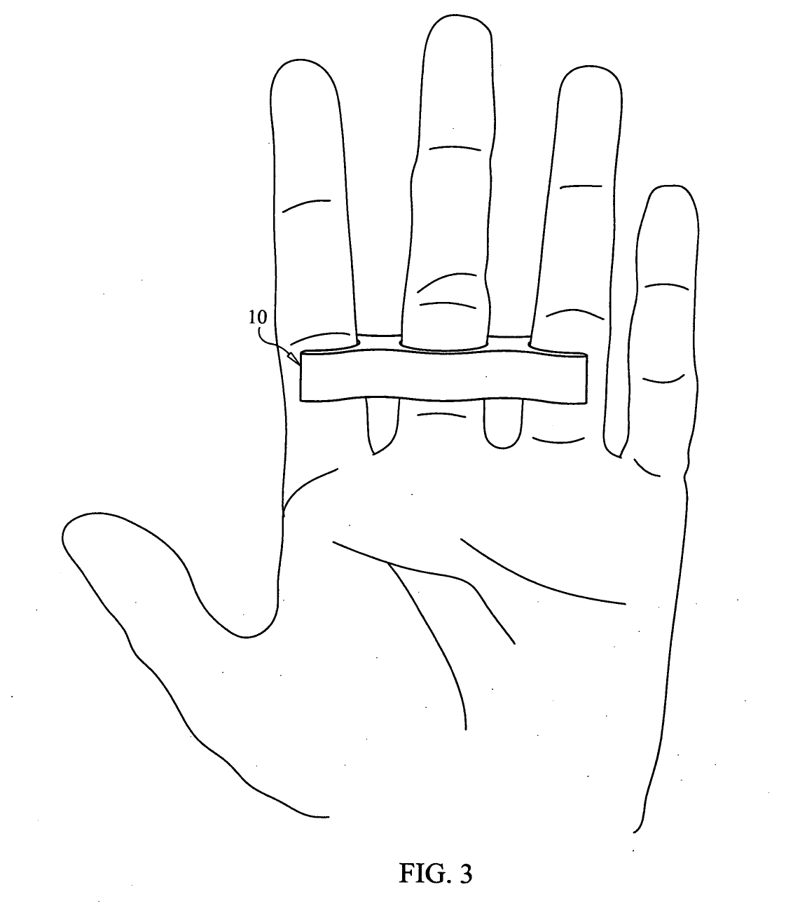

With reference to the drawings, FIGS. 1-3 depict the preferred embodiment of the instant invention which is generally referenced as a therapeutic finger brace and, or by numeric character 10. The instant invention 10 is designed to spread one's fingers for someone who has had an accident or ailment that causes them to merge and, or overlap.

With reference to FIGS. 1-3, the therapeutic finger brace 10 comprises a main body 12 defining a center aperture 14, a first arcuate end 16, a second arcuate end 18, an upper contoured side 20 and a lower contoured side 22. The center aperture 14 is designed for receiving a finger, such as the middle finger, while the first and second arcuate ends 16, 18 are adapted for receiving adjacent fingers. The upper contoured side 20 and lower contoured side 22 have an ergonomic design for comfort and facilitating use of the hand while wearing the therapeutic finger brace 10. The therapeutic finger brace 10 is preferably made from plastic. The center aperture 14, arcuate ends 16, 18 and contoured upper and lower sides 20, 22 are preferably smooth and devoid of sharp edges for comfort.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What is claimed is:

1. A therapeutic finger brace device device, said device comprising:
a main body;
a center aperture defined by said main body;
a first arcuate end;
a second arcuate end;
an upper contoured side; and
a lower contoured side.

2. A device as recited in claim 1, wherein said first arcuate side is curved.

3. A device as recited in claim 1, wherein said upper contoured side comprises two curves.

4. A device as recited in claim 1, wherein said lower contoured side comprises two curves.
